(19) United States
(12) Patent Application Publication
(10) Pub. No.: US 2018/0071067 A1
(43) Pub. Date: Mar. 15, 2018

(54) Medical Glove with an Area Suitable for Mixing Substances

(71) Applicant: Charles Sutera, III, Somerville, MA (US)

(72) Inventor: Charles Sutera, III, Somerville, MA (US)

(21) Appl. No.: 15/265,834

(22) Filed: Sep. 14, 2016

(51) Int. Cl. A61C 19/00; A61B 42/10; A41D 19/00; A61B 50/20

(52) U.S. Cl. CPC A61C 19/006; A61B 42/10; A41D 19/0082; A41D 2500/52; A61B 50/20; A41D 2500/40; A41D 19/0024

(57) ABSTRACT

A medical glove was designed to improve efficiency of operators in a variety of professions by combining the need for a mixing pad and/or dappan dish with a personal protective glove. The medical glove enables an operator to safely and ergonomically mix a variety of substances on their personal protective glove. The goal of the device is to increase procedural efficiency, increase working ergonomics, and increase protective safety of operators compared to current available products without decrease of overall glove tactile ability or comfort. The device provides an improved method compared to existing methods for ergonomic and safe placement and mixing of agents prior to placement in the working field.

### CROSS REFERENCE TO RELATED APPLICATIONS

The application claims the benefits of provisional patent application No. 62/276,943 filed Jan. 10, 2016 by the present inventor.

### FIELD OF THE INVENTION

The present invention relates generally to medical and dental personal protective equipment, and more specifically to medical surgical and exam gloves used by healthcare providers, researchers, and technicians

### BACKGROUND OF THE INVENTION

Many professionals in a variety of industries (e.g. medical, dental, scientific research, automotive, manufacturing) are required to use personal protective equipment such as medical grade exam or surgical gloves to prevent the operator from contacting hazardous chemicals, irritants, and/or bodily fluids. The most preferred gloves by operators are generally an ideal balance of several characteristics: protection from pathogens and caustic agents, tactility, ergonomics when working, and minimal risk of allergic reactions.

Health professionals wearing personal protective gloves commonly mix substances or transfer agents from a mixing pad to a working field. A common method typically requires material to be transferred by an assistant from a stock container to a separate mixing pad for preparation, then from the assistant to an operator, and finally to the working field. The traditional system is inefficient. A separate mixing pad requires multiple working spaces, multiple steps, is prone to cross contamination, and often requires working time by both an operator and an assistant.

To increase efficiency and ergonomics many healthcare professionals often mix substances or dispense substances on the back of their gloves until they are needed in the working field. For example, during a composite restoration procedure, dentists often place bonding agent on their gloves and dip their instrument into the bonding agent as a lubricant to prevent composite from sticking to their instruments. The location of utilizing the back of one hand to assist the other hand is convenient and ergonomic to access when working in the mouth. In a second example, a dentist may dispense a small amount of automix impression material from a mixing tip to the back of their glove to ensure complete mixing of the material prior to dispensing into the working field. The concept of utilizing a portion of a medical glove as a mixing pad has an ergonomic benefit, may increase efficiency, reduces need for assistance, and improves time management for operators. There are, however, disadvantages with the off label use of traditional medical gloves as mixing pads.

Although traditional medical gloves are successful at preventing transfer of infectious disease, they may exhibit permeability of chemicals of small particle size when left on the glove for an extended period of time. Methyl methacrylate (MMA) is an example of a common active agent used by many professionals, and is an ingredient in the bonding agent many dentists place on the back of their gloves as stated in the example in the preceding paragraph. MMA is also used to make hearing aids, joint prostheses, dentures, and is common in many adhesives. In a study in 2003 by Lorronoth R C et al, “Permeability of different types of medical protective gloves to acrylic monomers,” the researchers sought to determine the breakthrough time (BTT, min) of medical gloves as a measure of protection to chemicals commonly used by dentists and orthopedic surgeons (MMA, EGDMA and 1,4-BDMA). The study showed that “the smallest monomer MMA permeated within 3 minutes through all glove materials,” and concluded that “the breakthrough time (based on permeation rate) cannot be regarded as a ‘safe limit.” Moreover, the agent MMA as described in the above example contains serious health concerns. In another study completed in 2011 by Diaz J H, titled “Proportionate cancer mortality in methyl methacrylate-exposed orthopedic surgeons compared to general surgeons,” the researchers concluded that “MMA-exposed healthcare workers may be at increased risks of untimely deaths from site-specific malignancies,” such as esophageal cancer and myeloproliferative malignancies. MMA is just one example of a hazardous agent which has been found permeable across traditional medical gloves. U.S. Pat. Nos. 4,901,372 and 5,335,373 describe protective medical gloves and their use. Although it is possible to place and/or mix substances on the surface of said medical gloves, the permeability of common exam and surgical gloves could place the operator at risk when used in this manner. One may suggest to simply increase the overall thickness of the medical glove to reduce permeability. However, this is not beneficial either as it would reduce comfort and tactile ability of the operator.

The presented invention comprises a modified medical exam or surgical glove that is generally ergonomic, economical, safe, and supports an efficient method for holding and/or mixing substances by clinicians and technicians.

### SUMMARY OF THE INVENTION

In accordance with one embodiment, the placement or mixing of agents comprises a medical glove with an area suitable for placement or mixing substances similar to a mixing pad approximately positioned on the ventral or dorsal surface which may be utilized by healthcare providers.

Accordingly several advantages of one or more aspects are as follows: to provide a modified medical glove that enables a healthcare worker to temporarily place or mix agents via a mixing pad area which is relatively ergonomic, is relatively inexpensive, relatively comfortable, relatively resistant to tearing, and is relatively impermeable to commonly used chemical and agents by said healthcare worker. Other advantages of one or more aspects will be apparent from a consideration of drawings and ensuing description.

### BRIEF DESCRIPTION OF THE SEVERAL VIEWS OF THE DRAWINGS

FIG. 1 is a dorsal view of a glove demonstrating an embodiment.

FIG. 2 is a ventral view of a glove demonstrating an embodiment.

### DESCRIPTION OF EMBODIMENTS

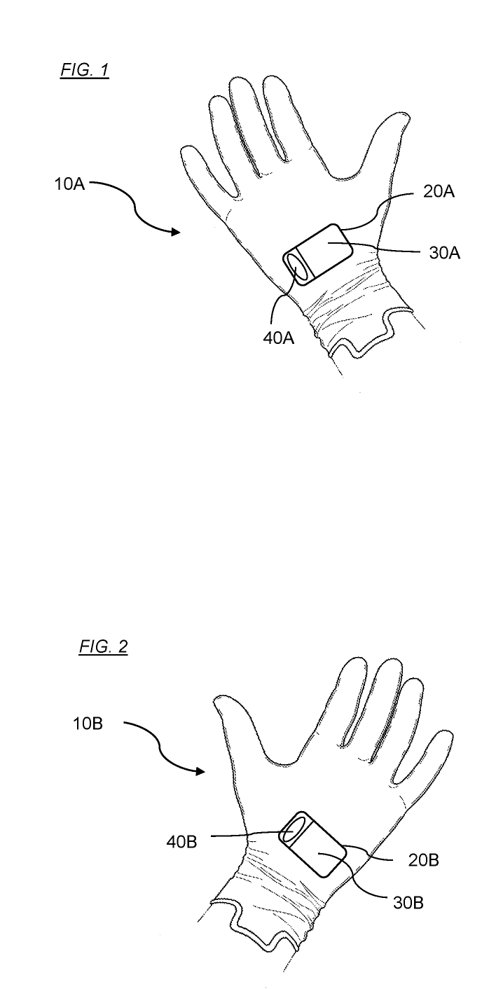

One embodiment of the medical glove [10A, 10B] is illustrated in FIG. 1 (dorsal view), and FIG. 2 (ventral view). The said medical glove [10A, 10B] comprises one or a plurality of demarcated areas suitable for placement or mixing substances [20A, 20B] which allow a healthcare provider to relatively safely place or mix agents on the working surface [30A, 30B].

In one embodiment the said medical glove [10A, 10B] comprises a nitrile rubber material and the said demarcated area suitable for placement or mixing substances [20A, 20B] is of sufficient thickness of nitrile rubber compared to the body of the glove such that the working surface [30A, 30B] has characteristics suitable for placement or mixing of agents such as acceptable resistance of permeability to said agents and acceptable resistance to tearing when mixing said agents in comparison to the body of said medical glove [10A, 10B]. The said medical glove [10A, 10B] and said demarcated area suitable for placement or mixing substances [20A, 20B] are described as comprised of nitrile rubber, however, the body of the medical glove [10A, 10B] may comprise any materials suitable for medical gloves and the demarcated area suitable for placement or mixing substances [20A, 20B] may comprise any materials which are relatively suitable for placement or mixing of agents by a healthcare provider. In one embodiment, the working surface [30A, 30B] may comprise an indentation portion [40A, 40B]. Healthcare providers may utilize said indentation portion [40A, 40B] in a function similar to a dappen dish or mixing well.

In one embodiment, the preferred location of said demarcated area suitable for placement or mixing substances [20A, 20B] is in an ergonomic area which does not compromise operator comfort or tactile ability such as the back of the hand on the lower third between the knuckles and wrist on the non-dominant hand, which also allows an ergonomic area when worn on the reciprocating dominant hand, the lower third of the palm below the pivot of the knuckles. The medical glove [10A, 10B] may be made in a plurality of varieties to accommodate right and left hand dominant preferences in regards to the positioning of the demarcated mixing pad area [20A, 20B].

The manner in which a dental professional uses the medical glove is during a medical, dental, or research related procedure which requires the use of both a personal protective medical glove and the use of a mixing pad for agents needed for the procedure. The operator wearing the glove may utilize the working surface [30A, 30B] within the demarcated area suitable for placement or mixing substances [20A, 20B] to place or mix agents common to their industry relatively safely and properly.

The presented embodiments of the device enable an improved method compared to existing methods for placement or mixing of agents needed in the working field in the operatory. The medical glove [10A, 10B] enables a method which is more ergonomic for the healthcare providers as the working surface [30A, 30B] becomes a wearable mixing pad in a convenient proximity for accessing required agents and delivering said agents to the working field of the procedure. The method enabled by the wearable mixing pad of the said medical glove [10A, 10B] furthermore serves to improve ergonomics as it frees a healthcare provider's hand or their assistance's hand which may then become available to hold a different item or instrument which may also be needed in the working field. Moreover, the said medical glove [10A, 10B] has a safety benefit. Many healthcare providers currently dispense agents to mix on their gloves, or bleed impression material from automix syringes onto the back of their gloves to ensure complete mixture prior to dispensing in the working field. Based on the research presented in the background section, the healthcare providers may potentially be placing themselves at risk by dispensing agents on traditional medical gloves without a demarcated area suitable for placement or mixing substances [20A, 20B].

The said medical glove [10A, 10B] may have an environmental benefit in regards to limiting waste produced in the healthcare profession. With introduction and adoption of the medical glove [10A, 10B] in the industry, medical glove waste theoretically should remain approximately undisrupted, however, waste of conventional separate mixing pads should theoretically decrease as use of said medical glove [10A, 10B] becomes more widespread. Accordingly, the reader will see the various embodiments provide medical professionals an ergonomic, fast, and safe method for placement or mixing of agents needed for the working field, as well as a potential environmental benefit.

Although the description above contains much specificity, these specifications should not be construed as limiting the scope of the embodiments, but as merely providing illustration of some of the several embodiments. For example, The demarcated area suitable for placement or mixing substances [20A, 20B] and working surface [30A, 30B] may be positioned, shaped, sized, textured, branded and/or textured in a variety of ways so long as the said characteristics allows for a healthcare provider to utilize the working surface [30A, 30B] to place and/or mix agents common to their industry relatively safely and properly. Moreover the medical glove [10A, 10B] could be manufactured to accommodate different shapes, and sizes of human hands.

Thus, the scope of the embodiments should be determined by the appended claims and their legal equivalents, rather than by only the examples given.

1) A medical glove comprising:
an area or plurality of areas suitable for placing and/or mixing substances on a portion of either the ventral or dorsal surface
wherein said area has relatively appropriate size, resistance of permeability, and tear strength such that a healthcare provider may use the working surface of said area to place and/or mix substances relatively safely and properly.

2) The medical glove of claim 1, wherein said medical glove comprises nitrile rubber.

3) The medical glove of claim 1, wherein said medical glove comprises latex rubber.

4) The medical glove of claim 1, wherein said medical glove comprises neoprene.

5) The medical glove of claim 1, wherein said medical glove comprises vinyl.

6) The medical glove of any one of claims 1 to 5, wherein said area is of greater thickness than any other region of the glove.

7) The medical glove of any one of claims 1 to 6, wherein said area is positioned distal to the wrist and proximal to the knuckles of the hand.

8) The medical glove of any one of claims 1 to 7, wherein said area has an indentation portion or plurality of indentation portions suitable to serve as a mixing well or dappen dish.

9) The medical glove of any one of claims 1 to 8, wherein said area comprises rubber.

10) The medical glove of any one of claims 1 to 8, wherein said area comprises plastic.

11) The medical glove of any one of claims 1 to 8, wherein said area comprises paper.

12) The medical glove of any one of claims 1 to 8, wherein said area comprises silicone.
